(19) United States
(12) Patent Application Publication
(10) Pub. No.: US 2016/0270820 A1
(43) Pub. Date: Sep. 22, 2016

(54) SELF PEDICURE FOOT COVERING AND METHOD OF USING SAME

(71) Applicants: Tim Helton, Cloverdale, IN (US); Dolly Helton, Cloverdale, IN (US)

(72) Inventors: Tim Helton, Cloverdale, IN (US); Dolly Helton, Cloverdale, IN (US)

(21) Appl. No.: 14/658,319

(22) Filed: Mar. 16, 2015

(51) Int. Cl. A61B 17/54; A41B 11/00; A45D 29/04

(52) U.S. Cl. CPC A61B 17/54; A45D 29/04; A41B 11/00

(57) ABSTRACT

A self pedicure foot covering includes a sock with an outer surface that is covered by outwardly abrasive areas separated by an elastic area. The outwardly abrasive areas include an instep patch, a toe patch, a heel patch, a left side patch and a right side patch that are arranged in a pattern that is symmetrical about a plane that bisects the sock. The elastic area has a distribution that allows the sock to fit a continuum of different sized feet, and the symmetrical pattern allows the sock to equally fit a left foot and a right foot. The different abrasive area patches have different grit sizes.

### TECHNICAL FIELD

The present disclosure relates generally to self administered pedicures, and more particularly to a sock with outwardly abrasive areas.

### BACKGROUND

Many individuals are limited in their ability to reach their feet to provide themselves a pedicure. For instance, for some individuals sanding toenails or exfoliating dry skin from other surfaces of their feet can be difficult to impossible due to limited flexibility, hand injuries or another reasons known in the art.

The present disclosure is directed toward one or more of the problems set forth above.

### SUMMARY

In one aspect, a self pedicure foot covering includes a sock with an outer surface covered by outwardly abrasive areas separated by an elastic area in a symmetrical pattern about a plane that bisects the sock. A combined area of the outwardly abrasive areas is many times greater than the elastic area. The outwardly abrasive areas include an instep patch, a toe patch, a heel patch, a left side patch and a right side patch. The instep patch, the toe patch and the heel patch are bisected by the symmetry plane. The left side patch and the right side patch are located on opposite sides of the plane. The instep patch is smoother than the right side patch and the left side patch. The left side patch and the right side patch are smoother than the toe patch, which is smoother than the heel patch. The elastic area has a distribution that allows the sock to fit a continuum of different sized feet. The symmetrical pattern allows the sock to equally fit a left foot and a right foot.

In another aspect, a method of performing a pedicure includes inserting one of a left foot and a right foot into the sock. The toe patch is rubbed against a first portion of the other foot. The heel patch is rubbed against the second portion of the other foot. The left side patch and the right side patch are rubbed against third and fourth portions, respectively of the other foot. The instep patch is rubbed against a fifth portion of the other foot. The sock is then removed from the first foot, and then the other foot is inserted into the sock. Different portions of the first foot are rubbed with the toe patch, heel patch, left side patch, right side patch and the instep patch respectively.

### BRIEF DESCRIPTION OF THE DRAWINGS

FIG. 1 is a perspective view of a self pedicure foot covering according to the present disclosure;

FIG. 2 is a top view of the self pedicure foot covering of FIG. 1;

FIG. 3 is a left side view of the self pedicure foot covering of FIG. 2;

FIG. 4 is a right side view of the self pedicure foot covering of FIG. 2;

FIG. 5 is a front view of the self pedicure foot covering of FIG. 2;

FIG. 6 is a backside view of the self pedicure foot covering of FIG. 2;

FIG. 7 is a bottom view of the self pedicure foot covering of FIG. 2;

FIG. 8 is a schematic view of a spectrum of different sized feet wearing the self pedicure foot covering of FIGS. 1-7;

FIG. 9 is a schematic view of a user with the self pedicure foot covering of FIG. 1 on the right foot while rubbing a portion of the left foot with the instep patch;

FIG. 10 is a view similar to FIG. 9 except showing the top side of the left foot toes being rubbed by the right side patch of the self pedicure foot covering;

FIG. 11 is the view similar to FIGS. 9 and 10 except showing the heel patch sanding the toenails of the left foot;

FIG. 12 is still another view of a self pedicure showing the left side patch exfoliating dead skin from the heel of the left foot; and

FIG. 13 is still another schematic view of a self pedicure showing the toe patch rubbing the underside of the opposite foot.

### DETAILED DESCRIPTION

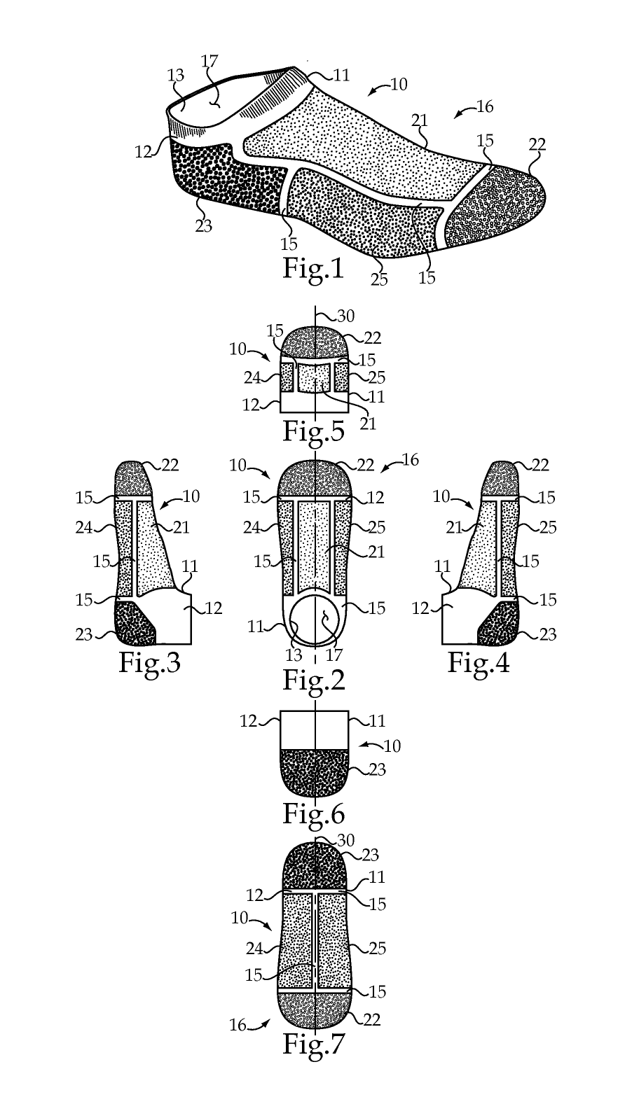

Referring initially to FIGS. 1-8, a self pedicure foot covering 10 includes a sock 11 with an outer surface covered by outwardly abrasive areas 20 separated by an elastic area 15. The outwardly abrasive areas 20 are arranged in a pattern 16 that is symmetrical about a plane 30 that bisects the sock 11. The combined area of the outwardly abrasive areas 20 is many times greater than the elastic area 5. In the context of the present disclosure, “many times greater” means at least three times. Together, the combined area of the outwardly abrasive areas 20 and the elastic area 16 equals the total area of the outer surface 12 of sock 11. The outwardly abrasive areas 20 include an instep patch 21, a toe patch 22, a heel patch 23, a left side patch 24 and a right side patch 25. The instep patch 21, the toe patch 22 and the heel patch 23 are bisected by plane 30. The left side patch 24 and the right side patch 25 are located on opposite sides of symmetry plane 30. The instep patch 21 is smoother than the right side patch 25 and the left side patch 24. Thus, if the roughness of the outwardly abrasive patch areas 20 was defined in a manner similar to sand paper, the smoother areas would have a higher grit size. The left side patch 24 and the right side patch 25 are smoother than the toe patch 22, which is smoother than the heel patch 23. Thus, in terms of grit size, one would expect the heel patch 23 to be characterized with the lowest grit size number. Although each of the outwardly abrasive areas 20 may have a uniform and predetermined grit size, some variation in roughness within a given patch 21-25 would also fall within the intended scope of the present disclosure.

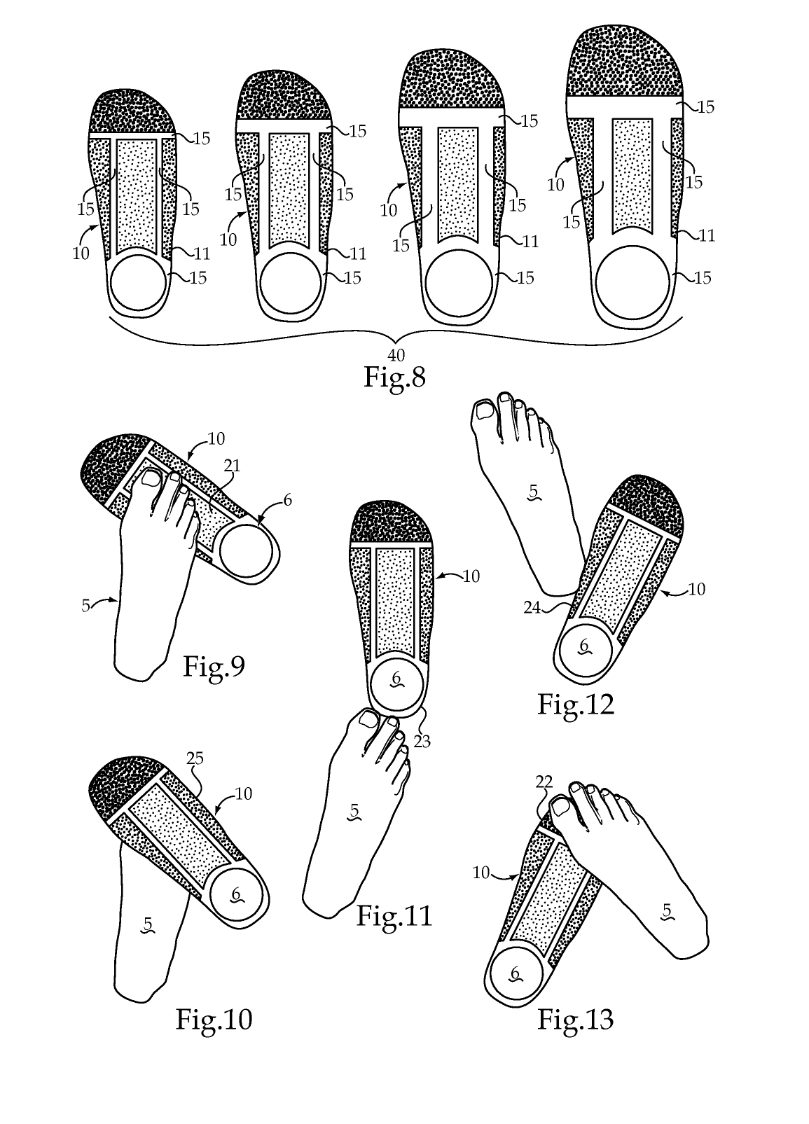

The elastic area 15 has a distribution that allows the sock 11 to fit a continuum of different sized feet 40, as best shown in FIG. 8. Although the outwardly abrasive areas 20 could be made to be themselves elastic, without departing from the scope of the present disclosure, the illustrated embodiment shows outwardly abrasive areas 20 having fixed areas, and relies upon elastic area 15 to stretch to accommodate larger sized feet. In addition, the symmetrical pattern 16 allows the sock 11 to equally fit a left foot and a right foot. Although not necessary, the entire inner surface 13 of sock 11 may be continuous cloth 17. Thus, the outwardly abrasive areas 20 may be separate patches of material attached to an underlying elastic cloth sock, or may otherwise be incorporated into the outer surface 12 of sock 11. In the illustrated embodiment, the continuous cloth 17 of the inner surface 13 may be elastic in order to provide for elastic area 15. In one specific embodiment, the outwardly abrasive areas 20 are comprised of emery cloth of pre-determined grit sizes. In other contemplated versions of the present disclosure, the self pedicure foot covering 10 could be made from a suitable disposable material, or could be made from a washable material well known in the art.

### INDUSTRIAL APPLICABILITY

The present disclosure is generally applicable to self administered pedicures. The present disclosure is specifically applicable to a one size fits all, left or right foot generic self pedicure foot covering for self administering a pedicure without the necessity to use one's hands.

Referring now in addition to FIGS. 9-13, a method of performing a pedicure with a self pedicure foot covering 10 includes inserting a first foot of a right foot 5 and a left foot 6 into the sock 11. The toe patch 22 is rubbed against a first portion of the other of the left foot and right foot. In the illustrated embodiment, the self pedicure foot covering is shown on the left foot 6, and FIG. 13 shows the toe patch 22 being rubbed against the underside of the toes of the right foot 5. The pedicure may continue by rubbing heal patch 23, which is the roughest, against a second portion of the left foot 6. For instance, FIG. 11 shows heel patch 23 being rubbed against the toenails of the right foot 5. Next, the left side patch 24 may be rubbed against a third portion of the other foot. For instance, FIG. 12 shows the left side patch 24 being rubbed against the heel area to exfoliate skin from the right foot 5. Next, the right side patch 25 may be rubbed against a fourth portion of the other foot. This is illustrated, for instance in FIG. 10 where the right side patch 25 is being rubbed over the top of the toes of the right foot 5. Next, the instep patch 21 may be rubbed against a fifth portion of the other foot. This is shown, for example in FIG. 9 where the instep patch 21 is being rubbed against the underside of the toe area of the right foot 5. After completing treatment of all the desired areas of the right foot 5, sock 11 may be removed from the left foot and inserted into the right foot. Thereafter, the individual user can rub different portions of the left foot 6 with the toe patch 22, the heel patch 23, the left side patch 24, the right side patch 25 and the instep patch 21, respectively in a manner similar to that shown in FIGS. 9-13, except with the left and right foot reversed.

The self pedicure foot covering 10 of the present disclosure allows a user, with limited ability to reach their feet with their hands, to apply a self pedicure limited only by the different relative positions they can obtain with their two feet. Thus, those persons with injuries or limited flexibility can exfoliate skin from their feet and sand their nails and other aspects of foot care using the self pedicure foot covering 10 of the present disclosure without necessity to resort to hand held tools and the like.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A self pedicure foot covering comprising:
a sock;
an outer surface of the sock being covered by outwardly abrasive areas separated by an elastic area in a pattern that is symmetrical about a symmetry plane that bisects the sock, and a combined area of the outwardly abrasive areas being many times greater than the elastic area;
the outwardly abrasive areas including an instep patch, a toe patch, a heel patch, a left side patch and a right side patch;
the instep patch, the tow patch and the heel patch being bisected by the symmetry plane;
the left side patch and the right side patch being located on opposite sides of the symmetry plane;
wherein the instep patch is smoother than the ride side patch and the left side patch;
wherein the left side patch and the right side patch are smoother than the toe patch, which is smoother than the heel patch;
wherein the elastic area has a distribution that allows the sock to fit a continuum of different sized feet; and
wherein the symmetrical pattern allows the sock to equally fit a left foot and a right foot.

2. The self pedicure foot covering of claim 1 wherein an inner surface of the sock is continuous cloth.

3. The self pedicure foot covering of claim 2 wherein each of the outwardly abrasive areas are emery cloth of a predetermined grit size.

4. A method of performing a pedicure with a self pedicure foot covering that includes a sock; an outer surface of the sock being covered by outwardly abrasive areas separated by an elastic area in a pattern that is symmetrical about a symmetry plane that bisects the sock, and a combined area of the outwardly abrasive areas being many times greater than the elastic area; the outwardly abrasive areas including an instep patch, a tow patch, a heel patch, a left side patch and a right side patch; the instep patch, the tow patch and the heel patch being bisected by the symmetry plane; the left side patch and the right side patch being located on opposite sides of the symmetry plane; wherein the instep patch is smoother than the ride side patch and the left side patch; wherein the left side patch and the right side patch are smoother than the toe patch, which is smoother than the heel patch; wherein the elastic area has a distribution that allows the sock to fit a continuum of different sized feet; and
wherein the symmetrical pattern allows the sock to equally fit a left foot and a right foot, and the method comprising the steps of:
inserting first foot of a left foot and a right foot into the sock;
rubbing the toe patch against a first portion of an other foot, which is an other of the left foot and the right foot;
rubbing the heel patch against a second portion of the other foot;
rubbing the left side patch against a third portion of the other foot;
rubbing the right side patch against a fourth portion of the other foot;
rubbing the instep patch against a fifth portion of the other foot;
removing the sock from the first foot and then inserting the other foot into the sock; and
rubbing different portions of the first foot with the toe patch, the heel patch, the left side patch, the right side patch and the instep patch, respectively.
